(19) United States
(12) Patent Application Publication
(10) Pub. No.: US 2002/0174462 A1
(43) Pub. Date: Nov. 21, 2002

(54) Ramie

(75) Inventor: John L. Baker, Naples, FL (US)

Correspondence Address: Scott M. Oldham, Oldham & Oldham Co., L.P.A., 1225 W. Market St., Akron, OH, 44313, US

(73) Assignee: Contex Industries, L.C.

(21) Appl. No.: 10/038,108

(22) Filed: Oct. 19, 2001

Related U.S. Application Data
(63) Continuation of application No. 09/319,197, filed on Jul. 28, 1999, now abandoned.
(60) Provisional application No. 60/032,203, filed on Dec. 2, 1996.

(30) Foreign Application Priority Data
Dec. 2, 1997 (US) PCT/US97/22259

(51) Int. Cl. A01H 5/00

(52) U.S. Cl. 800/323

(57) ABSTRACT

This invention relates to an improved variety of ramie which is more freeze and frost-resistant than previous varieties as well as having improved physical characteristics. The improved species of ramie matures faster on average, has a higher tensile strength, and most significnatly, has a finer average denier than prior art ramie.

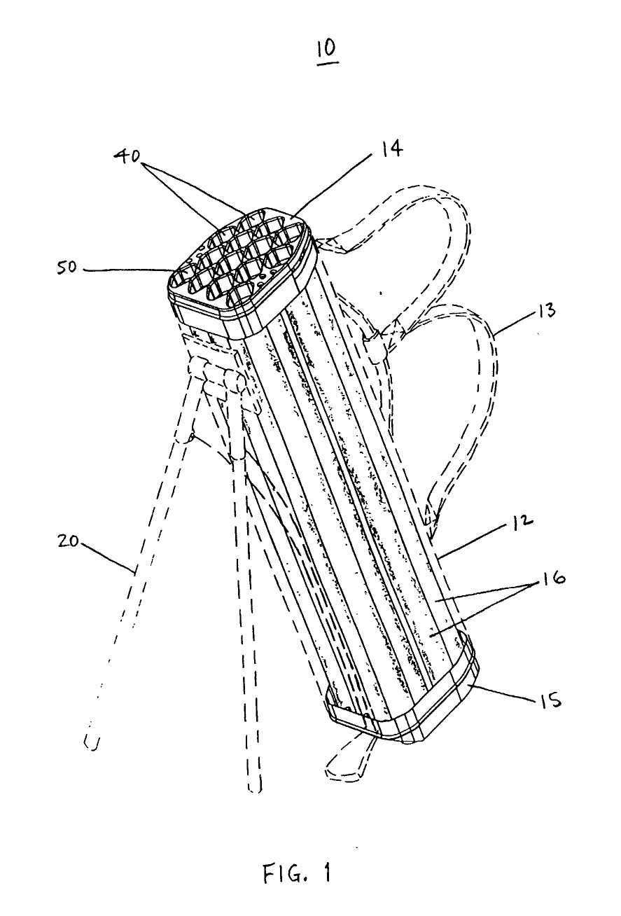

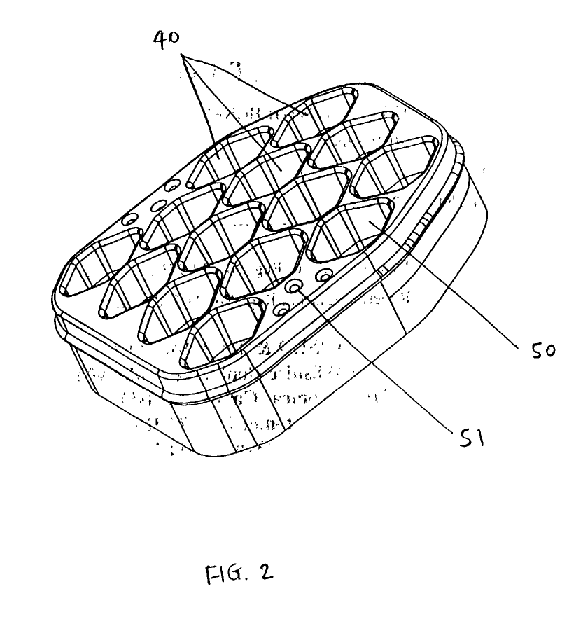

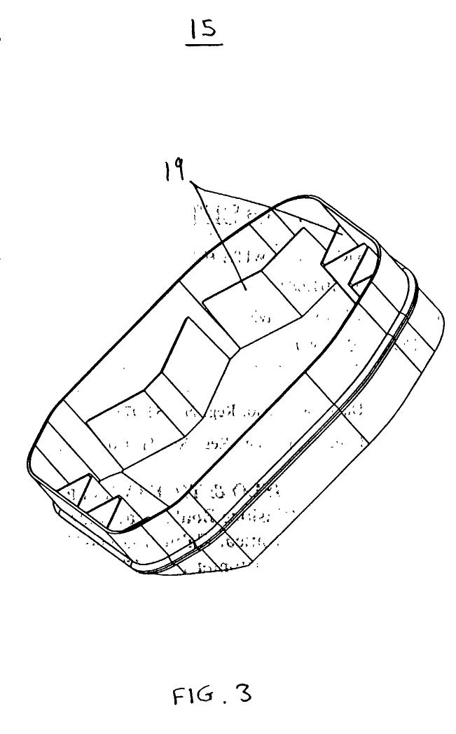

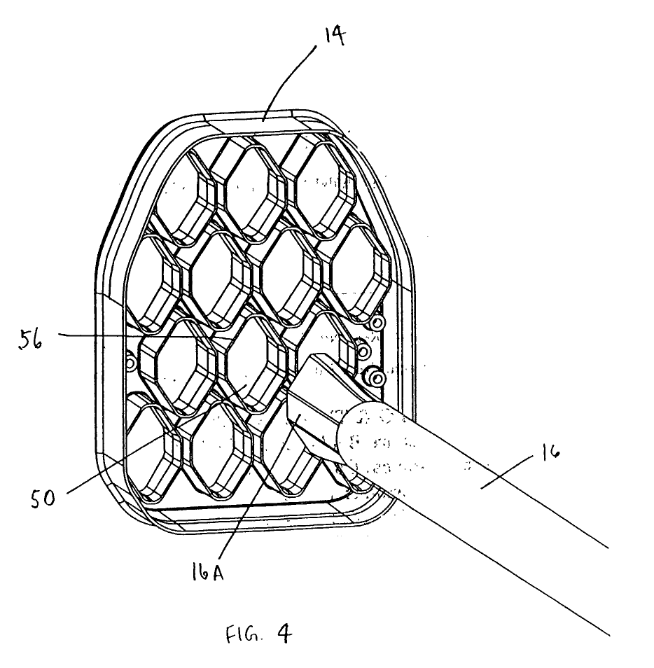

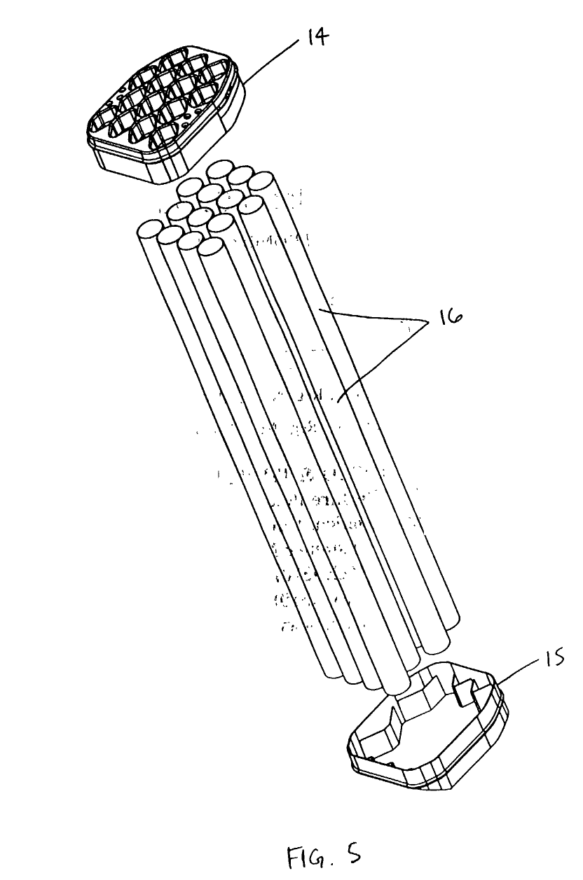

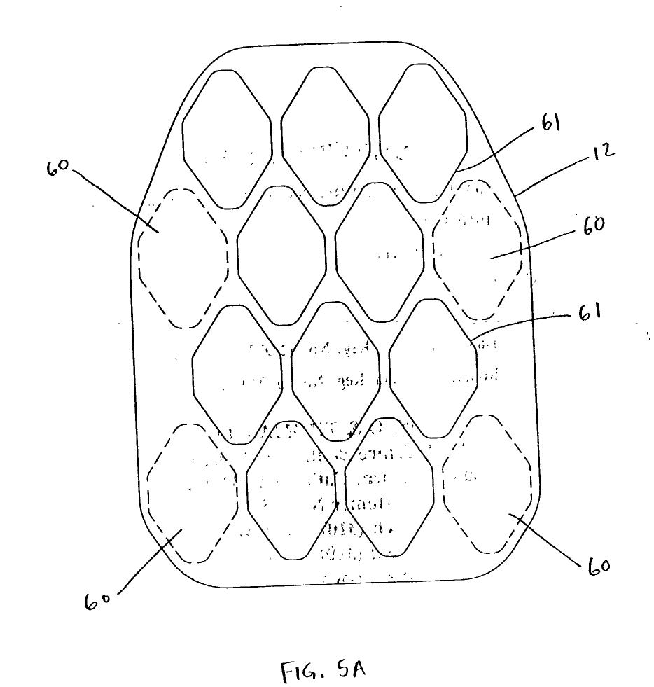

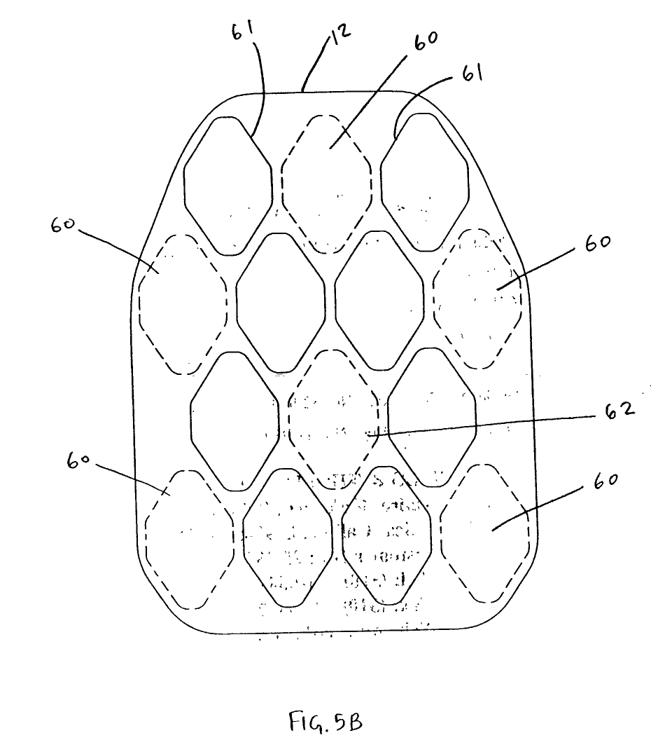

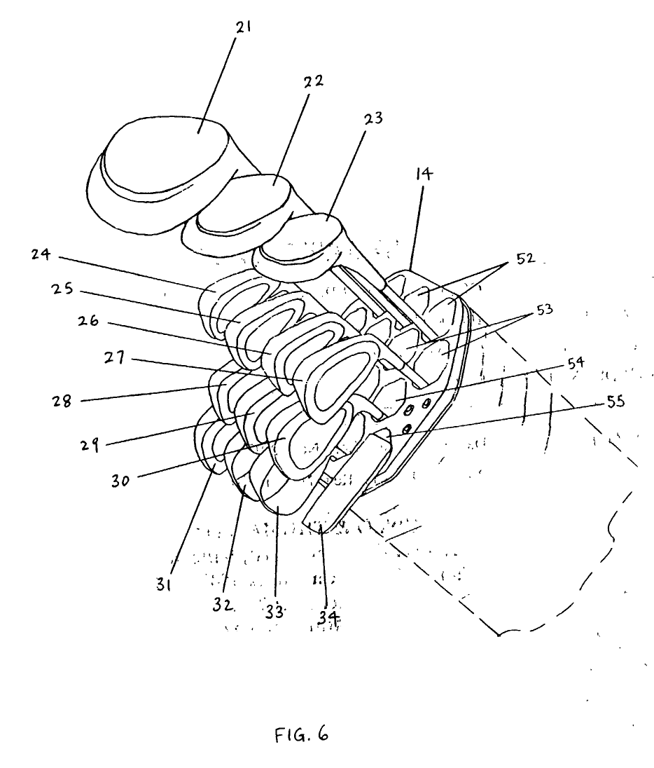

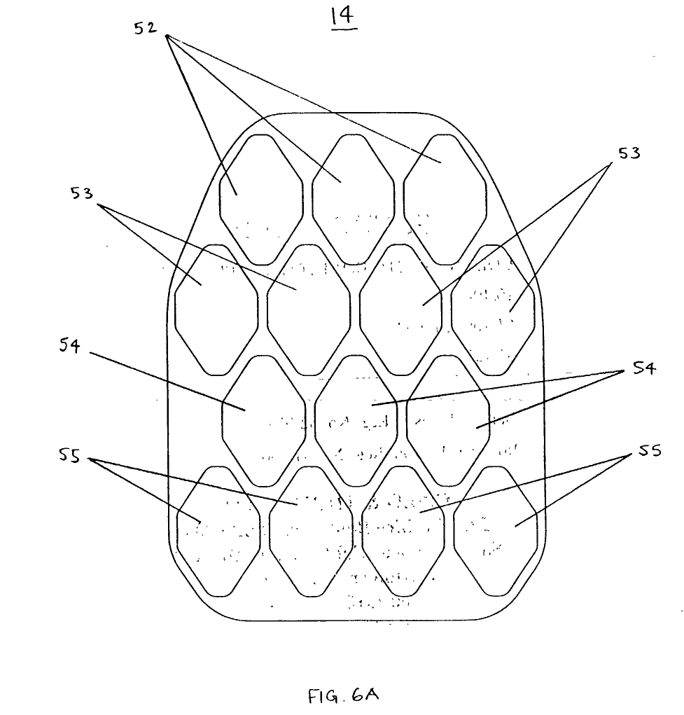

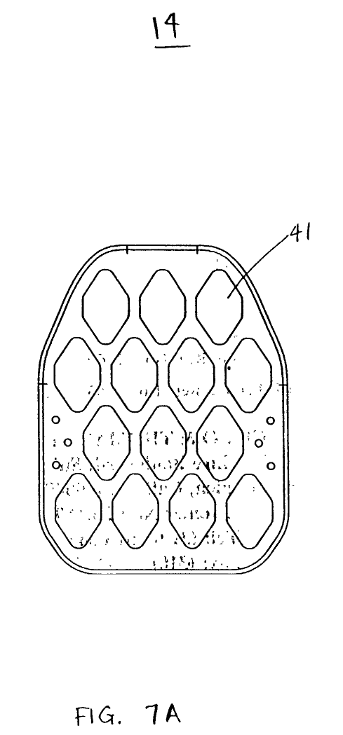

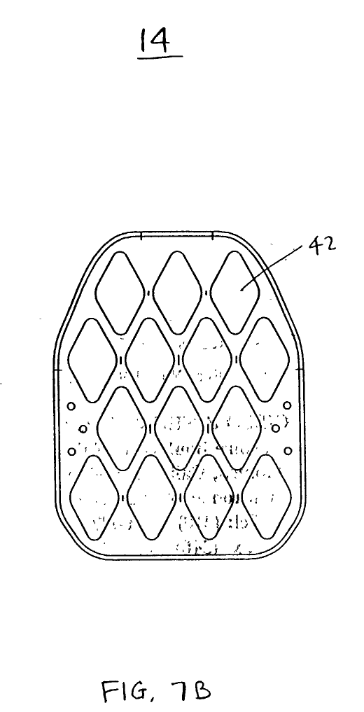

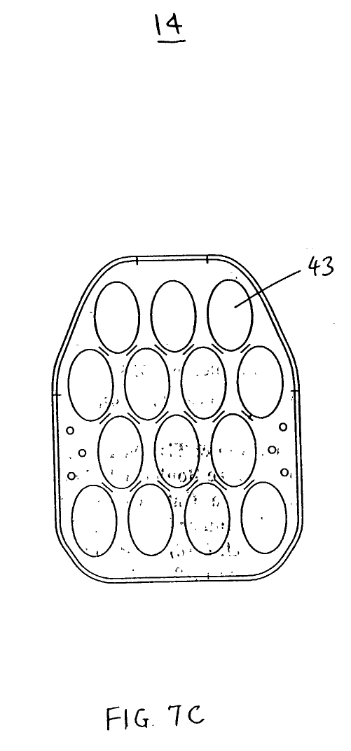

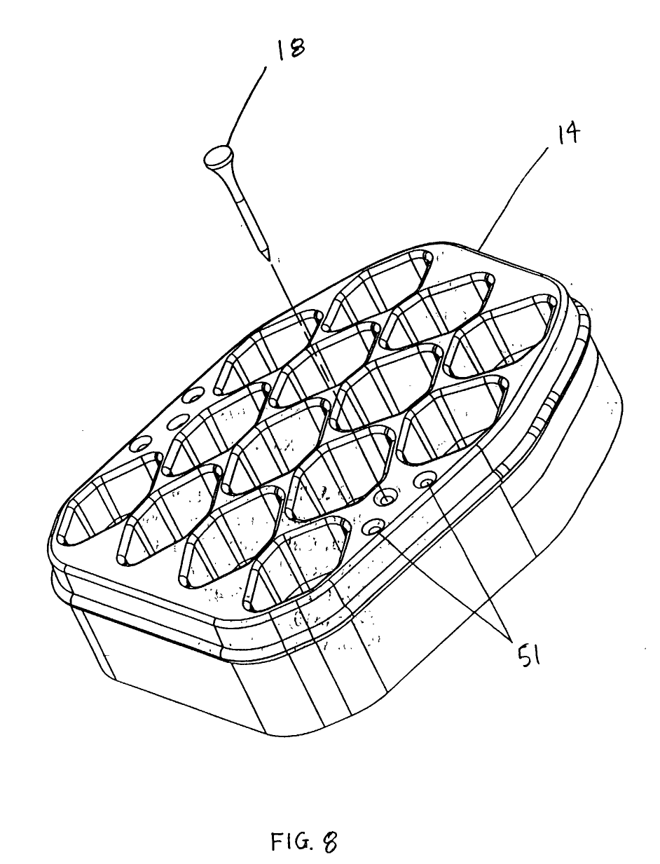

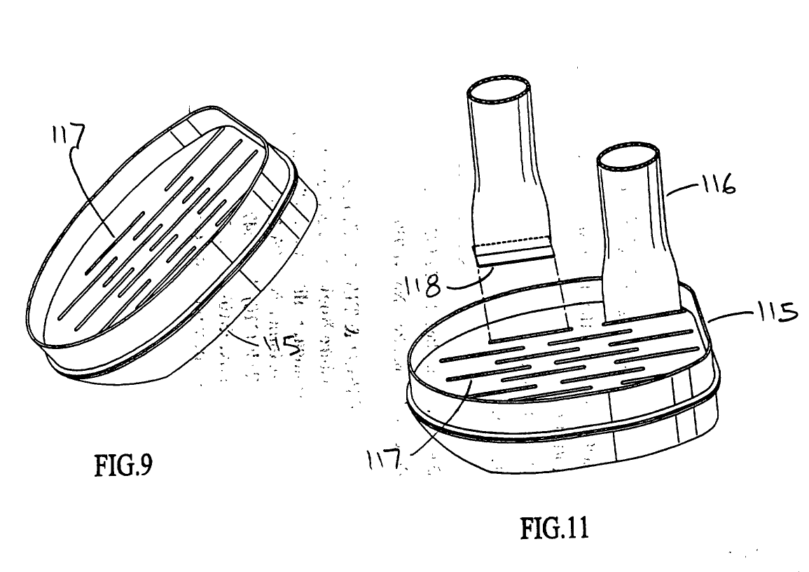

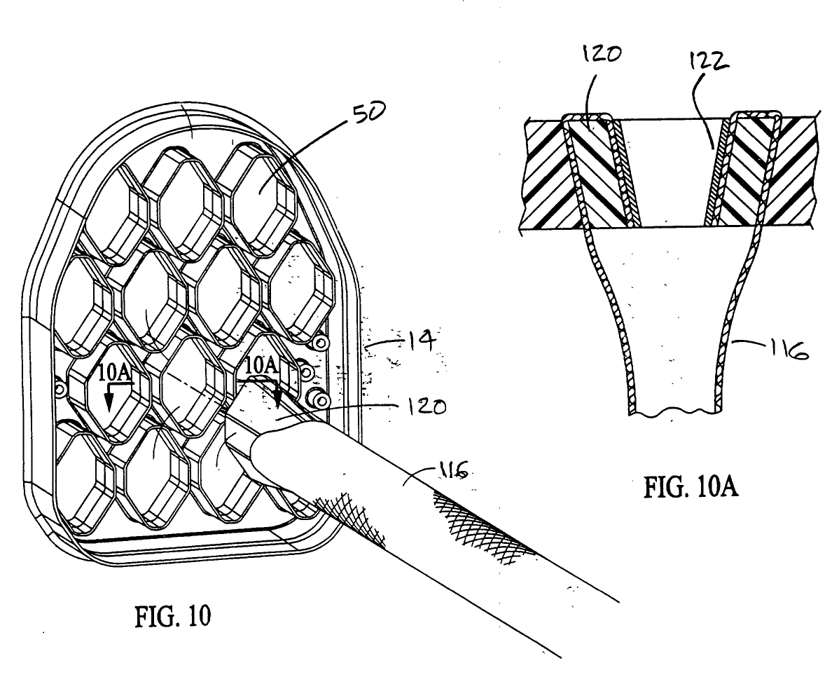

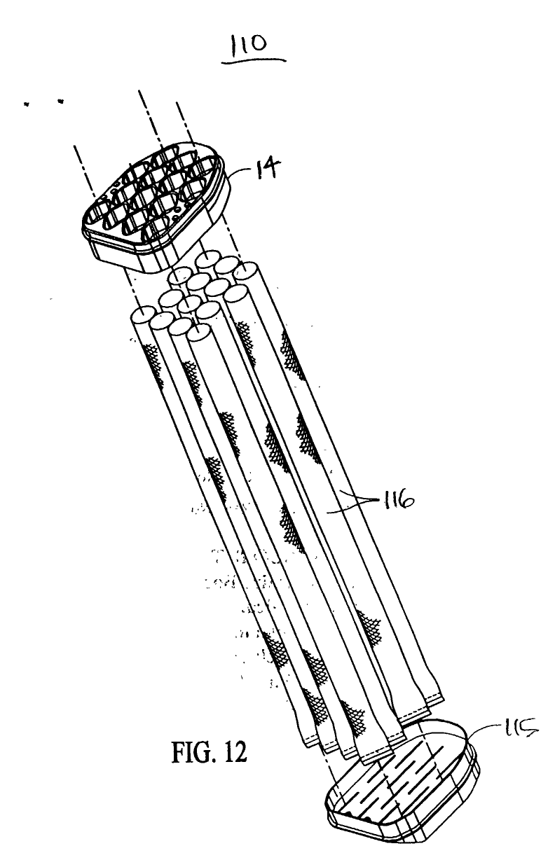

### TECHNICAL FIELD

This invention relates to an improved variety of ramie which is more freeze and frost-resistant than previous varieties as well as having improved physical characteristics.

### BACKGROUND OF THE INVENTION

Ramie is a bast fiber plant of the nettle family (Urticaceae) and probably originated in Asia. Ramie has been grown in China for centuries, hence the name “China Grass.” Agronomic studies have been conducted in the past in the United States and Europe on the fibrous and foligate properties of ramie. While there are several species of ramie, until the present invention, the Boehmeria nivea (L) Gaud, P.I. 87521, had represented the most promising species for fiber production.

Although ramie blends are used in apparel, (e.g., sweaters, dresses, shorts, etc), the ramie used in such blends is currently imported into the United States, there is presently, no ramie commercially produced in the United States. Ramie and kenaf had been grown in South Florida in the late 1940s and 1950s, but was not commercially successful for at least three fundamental reasons: (1) the introduction of synthetic fibers (e.g., rayon, nylon, etc) into the textile industry which were extremely cost-competitive with natural fibers such as ramie, cotton, wool, etc., due to the then, low cost of oil; (2) the inefficient and labor-intensive methods of harvesting, decorticating ramie (i.e., stripping the outer bark from the inner fiber), processing ramie (i.e., degumming the decorticated fibers) and stapling of ramie (i.e., the cutting of the approximately six foot decorticated fiber strands into commercially usable lengths); and (3) the requirement that the ramie be grown in tropical or subtropical regions of the world, thereby limiting ramie production to the extreme south Florida region of the United States.

In 1996, Congress enacted legislation which phased out the sugar cane grower/producer's price support subsidy administered by the United States Department of Agriculture, an action which will significantly impact Florida's industry, and in fact, will also be applicable to other sugar cane growing regions such as Louisiana, Texas and Hawaii. This “Fair Farm” Act, signed into law by President Clinton on Apr. 4, 1996, will eliminate price support systems over seven years, thereby systematically reducing the world market price of this commodity, and encouraging alternative land use. In Florida, one of the prime sugar cane growing regions lies south of Lake Okeechobee in the “Everglades Peat” (i.e., muck) areas. Environmental studies by various groups over the years has yielded the claim that the sugar cane industry has negatively affected this natural resource, and the Fair Farm Act contains financial provisions which are intended to work toward addressing this problem.

Today, the issues which prevented the commercialization of ramie production are no longer present. First, the price of oil has increased significantly, thereby removing the first impediment. Second, the inefficient and labor-inventive harvesting and processing steps have been automated through the developing of the patented Harvestor/Decorticator as described in U.S. Pat. No. 5,632,135, which decorticates and staples the harvested ramie, chemically treats the stapled fiber with a “wet bath” and separates the forage (i.e., leaves and plant bark and core) into separate bins in the field in one continuous harvesting action. And lastly, this invention addresses the remaining issue of the climatization of ramie, now permitting propagation and plant survival not only in north Florida, but also in latitudes including Georgia and Alabama, potentially extending as far north as North and South Carolina in the United States. This variety with improved cold weather resistance is a potential alternative crop to tobacco, cotton, soybeans, kenaf, etc.

Additionally, ramie plant by-products, e.g., bark, core and foliage, maybe suitable for use as animal feed with a 20-25% protein content, similar to alfalfa. The bark may also have applicability for particle board manufacture. Through the combination of genetic engineering which imparts increased frost-resistance thereby permitting growth in more temperature zones to that traditionally required (i.e., tropical or sub-tropical), coupled with changing market conditions and improved harvesting techniques, the improved ramie is poised to become a significant market force in the future.

### SUMMARY OF THE INVENTION

This invention relates to a more freeze and frost-resistant variety of ramie which permits its growing in climates other than tropical or sub-tropical. This increased climatization characteristic, one of the objectives of this work, has been imparted into ramie through genetic selection and engineering of the species. This new ramie species also exhibits a finer denier, which is a highly sought-after attribute and another objective of this invention.

These and other objects of this invention will be evident when viewed in light of the detailed description and appended claims.

### DESCRIPTION OF THE PREFERRED EMBODIMENTS

This invention will now be described in detail with referenced to preferred embodiments thereof. Throughout the specification, including the claims, compositions are given in percent by weight unless the contrary is expressly stated.

The key to this new variety of ramie is its climatization to more northern temperature zones, more specifically, its increased frost-resistance. In developing this superior strain, various rhizome stocks of ramie, including P.I. 87521 were exported to Costa Rica from the United States in 1981 for growing at various altitudes and in different soil conditions. The result of these efforts is P.I. 87521-X (BELLE RAMIE™), that prospers not only in Everglades peat, but in sandy loam soils, and in latitude zones as far north as Kentucky in the United States.

Ramie, Boehmeria nivea (L) Gaud, is a member of the Urticaceae or Nettle family, and is the most important species of this genus which is grown for the production of the long vegetable fibers found in the bast or inner bark of the plant. This fiber has a tensile strength greater than that of any of the other soft or bast fibers, is very resistant to deterioration when thoroughly degummed, and possesses a soft and lustrous property. These characteristics should and do make ramie fiber very valuable for many industrial uses. The raw fiber of commerce is frequently referred to as “China Grass,” because of the grass-like appearance of the hand-cleaned fibers which have been produced in China for centuries. The individual fibers are about 10-20 cm in length and have a cross-section diameter of about 20-70 microns.

The family Urticaceae is rich in fiber-bearing plants, but most of them have not been studied or utilized extensively. References in the literature mention numerous species belonging to 25 or more genera from which fiber has been extracted for use in native industries. Most of these references are concerned with plants native to southeast Asia and Africa. The genus Boehmeria represents a good-sized section of the Nettle family. The species of Boehmeria are mostly shrubs or trees, sometimes as much as 30 feet in height (B. excelsa), but many species are herbaceous perennials. The herbaceous species are similar in appearance and foliage to true nettles (Urtica) but differ in the fact that they lack stinging hairs and in the nature of the calyx. In Urtica, the calyx is composed of four essentially separate sepals in both the staminate and pistallate flowers. In Boehmeria, the calyx in the pistillate flower is tubular-shaped, often compound, merely 2-4 toothed at the apex, and encloses the achene; the staminate calyx is much the same as Urtica.

The genus Boehmeria is found in both hemispheres in both tropical and temperate regions. Only one well-defined species is known in the United States, Boehmeria cylindrica, which occurs as far north as Maine and Southern Canada. Boehmeria cylindrica is a perennial herb, grows 1-3 feet tall and is found in moist soils.

The leaves of B. nivea are heart-shaped, about 2-5 inches in width, with finely serrated margins, and devoid of the stinging hairs which many other members of the nettle family possess. The color of the upper side of the leaf is green, and silvery white on the under surface. The stems are slender, about 7-20 mm in diameter and frequently attain a height of 6-7 feet. The flowers are greenish-white in color and are borne in declinate clusters in the axils of the leaves. Pistillate and staminate flowers are generally found on the same stalk; the former on the upper part of he stalk and the latter on the lower part of the stalk. The seeds, which are very small, ovate in shape and enclosed in a persistent calyx, are produced in very large quantity.

The plant has a regular dimorphic root system consisting of bulbous or faciculate storage roots, and reproductive or rhizome roots. In the early stages of growth, many plants generally produce only storage roots. The storage roots have no buds, and consequently in commercially practice, only the rhizome roots can be used for planting stock.

In 1946, seed of the ramie variety, P.I. 87521, was planted in flats in the greenhouse at the Everglades Experiment Station. More than 1600 seedlings obtained from these plantings were set out on Everglades peat soil in a field on the Newport ramie plantation in August, 1946. About six months later, 43 of these seedlings which showed promising vegetative characteristics, were selected for further observation. In the spring of 1947, these seedlings were planted at the Fiber Nursery at the Everglades Experiment Station from vegetative material. During the 1947 and 1948 seasons, observations and tests were made on the plantings of these promising selections at the two locations in order to determine whether they actually possessed the qualities necessary for good fiber production. Particular attention was paid to the determination of fiber content and quality, as well as other plant characteristics, such as height, size and density of stalks, and time of the maturity of the plant, as indicated by initial flower development.

Twenty-seven of the selected seedlings, representing 1.6% of the total plants originally obtained, showed fiber contents greater than that of the commercially grown variety, P.I. 87521, which contained 2.53% mechanically recoverable fiber on a green plant weight basis under the conditions of these tests. The highest fiber content obtained form the selections was 3.29% and the lowest was 1.90% fiber. These results seem to indicate that there is considerable scope for increasing the fiber content of ramie by means of selection from the large and comparatively unexplored pool of variation, which should be obtainable from this heterozygous plant. The combined results for the 1947 and 1948 of the ten best fiber yielding selections harvested when mature are shown in Table 1, below as well as data for the commercially grown variety, which is included for comparative purposes.

TABLE 1

| | | | Tensile Strength of |
|---|---|---|---|
| | Fiber Content | Total Height | degummed fiber-½″ break |
| Ramie | (%) | (in) | (lbs per sq in) |
| Experimental | 3.04 | 66 | 40,300 |
| P.I. 87521 | 2.53 | 67 | 39,500 |

In an effort to increase the climate resilience of the ramie plants, various P.I. 87521 plants as well as all of the remaining varieties of ramie then on the experimental plot from the University of Florida's Everglades Experimental Station and were taken to Costa Rica in 1981 and planted at sea level. Costa Rica was chosen due at least in part to its terrain and year-round growing season. At harvest time after maturation of the plant (approximately 24 months), a subset of the crop was selected based on the following criteria which included height, thickness of the stem, percentage of fiber per stem, growth pattern within a defined period from cutting, fiber fineness and percentage of gums per fiber count. These selected plants were transplanted both at sea level and increasingly higher elevations (3,000-4,000 feet) through 1987 and allowed to grow and mature. A subset of that crop was re-introduced into the United States in December 1995. Through this planting of experimental crops at various altitudes and soil conditions, a cold and frost resistant strain of ramie was produced from rhizome seed stock.

The chemical composition of the tops and leaf material of the ramie plant grown on the organic soils of the Florida Everglades indicates that it can provide a highly nutritious livestock feed. A typical chemical analysis of ramie tops is given in Table 2 below.

TABLE 2

| Component | Percentage |
|---|---|
| Moisture | 10.8 |
| Ash | 14.2 |
| Protein | 25.5 |
| Ether Extract | 5.5 |
| Crude Fiber | 12.2 |
| N-free Extract | 44.6 |
| Carotene | 68.4 mg per lb. |
| Riboflavin | 7.7 mg per lb. |

Protein values range from 20-24% for the tops and as high as 28% for the leaves taken alone. It is estimated that a yield of about 14 tons per acre of top material (leaf and stem), containing about 80-82% moisture, can be obtained from three harvests of plants in a year. The carotene and riboflavin content of the tops is also high. Therefore, 68-70 mg per lb for carotene and 7.7-8.0 mg per lb for riboflavin compares very favorably with average values for these components in alfalfa leaf meal.

Ramie variety, P.I. 87521, is distinguishable from other varieties of ramie because of the inherent characteristics of this variety. It is resistant to insects and is resistant to diseases normally associated with fibrous plants. The quality of the fiber is acceptable for the U.S. textile industry with an average denier (fiber diameter) of 8. The growth cycle of this variety when mature, allows for four harvests a year in comparison to three in a semi-tropical climate.

Ramie variety P.I. 87521-X (BELLE RAMIE™) is distinguished from other varieties of ramie because of its unique characteristics. Like P.I. 87521, it is also resistant to insects and diseases. The fiber production quantity as to the percentage of fiber produced per stem makes it an economically viable crop and is greater than the quantity produced by P.I. 87521. The quality of the fiber is also acceptable for the United States textile industry and world market. Unexpectedly, it has a finer denier than the P.I. 87521 with an average denier of 6. The growth cycle allows for up to five harvests a year versus a normal three for a semi-tropical climate.

The varieties P.I. 87521 and P.I. 87521-X share the following unique features of the Boehmeria nivea (L) Gaud ramie species: (1) The presence of maculate hairs. Maculate hairs are a type of protective hair, which is one of the peidermal hairs special to ramie. It's remarkable difference is the shape, size, distribution and density of spots of maculate hairs for different varieties. Therefore, maculate hairs are one of the characters for identifying varieties and are of importance for plants to be in the classification. (2) The perianth does not open from an alabastrum to a mature achene for ramie female flowers. The blooming stage is determined by the morphological features of style, stigma, ovary and perianth. As utilized in this new variety, P.I. 87521-X, this feature is an important pattern of the ramie plant to cross-bread new varieties. (3) The style of the ramie pistil is developed from one side of the carpel and the stigma grows on the side of the style. The special structure of the ramie pistil is another special feature for the purpose of an angiosperm because of this kind of special structure. (4) The blooming mechanism of the ramie male flowers is actualized depending on the special structure and function of the filament of the stamen and is, therefore, different from that of any other angiosperm, which is an additional example oframie's uniqueness in the plant kingdom. (5) Ramie fiber in the stem originates in the pericycle and is a kind of complex fiber, which are individual fibers consisting of a series of fiber cells placed end to end. Applicant's studies have demonstrated that the view formerly held that the ramie fiber originated from pholem fiber is not so, and has established that it is, in fact, from the development of individual fiber.

Further characteristics which are variable, but still useful for a meaningful description of the variety, would include the following information shown in Table 3, as recognized by the United States Department of Agriculture under the Plant Varieties Protection Act.

TABLE 3

| Characteristics | P.I. 87521 | P.I. 87521-X |
|---|---|---|
| Plant Habit (spreading, | spreading | spreading |
| intermediate, compact) | | |
| Foliage (sparse, intermediate, | dense | dense |
| dense) | | |
| Stem Density | 5-10 sq. Ft | 6-12 sq. Ft |
| Stem Size | 2.5 cm. | 2.7 cm. |
| Stem Height | 2 m | 2.25 m |
| Growth (determinate, | determinate | determinate |
| intermediate, indeterminate) | | |
| Yield of green plant material | | |
| (lbs./per acre/per harvest) | | |
| Harvest #1 | 15,000 | 20,000 |
| Harvest #2 | 15,000 | 20,000 |
| Harvest #3 | 15,000 | 20,000 |
| Harvest #4 | 15,000 | 20,000 |
| Leaf Color | medium green | medium green |
| (greenish yellow, light green, | | |
| medium green, dark green) | | |
| Leaf Color (underside) | silver | silver |
| Maturity | 60 days (avg.) | 55 days (avg.) |
| Average Stem Size (diameter) | 2.5 mm | 2.5 mm |
| Stems per square foot (mature) | 5 | 6 |
| Mature Plant Height (m) | 2 | 2 |
| Stem (fiber content %) | | |
| Harvest #1 | 2.7 | 2.7 |
| Harvest #2 | 3.4 | 3.4 |
| Harvest #3 | 3.1 | 3.1 |
| Harvest #4 | 2.6 | 2.5 |
| Fiber Percentage after Degumming | 75% | 75% |
| Tensile strength of Degummed | 52,400 | 60,000 |
| Fiber-½″ break | | |
| (lbs per sq in.) | | |
| Average Denier | 8.02 | 6.0 |
| Grams of Fiber per Stem | 12.5 | 15 |
| Lignon percentage after | 2% | 2% |
| Degumming | | |
| Fiber Properties | | |
| Length (inches, 2.5% SL) | 5-8″ | 5-8″ |
| Elongation, EI (%) | 3% | 3% |
| Micronaire | 55 × 40 | 55 × 40 |
| Fineness | 200-300 NM | 200-300 NM |

Additional testing was performed in an air-conditioned laboratory maintained at 70° F. and a relative humidity of 6%. The testing equipment included a Scott Tester Model DH, a Scott Tester IP-4 (inclined plant principle), a flex testing machine and a wear testing machine, yielding values for tensile strength, shear strength, elongation or stretch, flex and wear. Approximately 550 samples from the P.I. 87521 series of varieties were tested. Good normal degummed ramie fiber generally tested around 60,000 pounds per square inch on the 1/2″ break made according to the procedure for testing cordage fibers. Ramie yarn samples tested show about twice the strength of cotton yarn samples tested in a similar manner. The shear strength of ramie is approximately 60% of its tensile strength. Ramie yarns tested stretch approximately 17% of the length to break, or ⅔ the stretch of similar cotton yarns tested at twice the load. The flex quality of ramie is influenced primarily by the degree of degumming and by the softening agent used in its processing. Undegummed ramie or partially degummed ramie has an extremely short flex life. However, well degummed ramie, which has been properly processed, has a very long flex life, and some yarn samples tested in comparison with cotton yarns have given as much as ten times the flex life of cotton. An average figure would be from 3-5 times that of cotton.

Ramie is a harsher fiber than cotton and is somewhat hard to the touch, comparing more with linen in this respect. This harshness is well demonstrated on the wear machine where two samples are rubbed against each other until one breaks in order to obtain something of a quantitative value for this quality. Ramie fiber tests quite low on this machine, and yarns show only about 20% of the wear life of similar cotton yarns. Nevertheless, a ramie yarn sample in this machine will cut through a cotton yarn sample in about 70% of the life off the cotton yarn sample with apparently no damage to the ramie yarn. This would indicate that ramie has a much greater wear resistance than cotton when the two fibers are subjected to a wear test against each other.

In comparative testing shown in Table 3, nearly identical yarn samples of cotton and ramie are reported.

TABLE 3

| | Ramie | Cotton | % of Cotton |
|---|---|---|---|
| Yarn Size | 10/6 m | 6/6 cc | |
| Weight (Mgs/M) | 615 | 655 | |
| Cross-Sectional Area | 0.00088 | 0.00094 | |
| (square inches) | | | |
| Breaking Strength (3″) | | | |
| Dry (lbs.) | 40.00 | 20.7 | 194 |
| Variation (%) | 21 | 15 | |
| Wet (lbs.) | 53.1 | 23.3 | 228 |
| Variation (%) | 33 | 13 | |
| Tensile Strength | | | |
| Dry (psi) | 45,400 | 22,000 | 206 |
| Dry (km) | 29.5 | 14.3 | 206 |
| Wet (psi) | 60,300 | 24,000 | 252 |
| Shear Strength | | | |
| Dry (lbs.) | 23.6 | 20.7 | 114 |
| % of Breaking Strength | 59 | 100 | |
| Wet (lbs.) | 32.0 | 22.0 | 146 |
| Increase due to wetting (%) | 36 | 6 | |
| Dry (psi) | 26,800 | 22,000 | 122 |
| Wet (psi) | 35,400 | 23,400 | 146 |
| Elongation (to break in 3″) | 0.52 | 0.81 | 64 |
| Wear Cycles | | | |
| Dry (SS) | 150 | 960 | 16 |
| Dry (SZ) | 190 | 900 | 21 |
| Wet (SS) | 160 | 200 | 80 |
| Wet (SZ) | 240 | 180 | 133 |
| Flex (cycles) | 347,000 | 122,000 | 284 |

Ramie fibers, when tested wet, show an increase in tensile strength and shear strength of approximately 40%, and there is an increase in the wear life of ramie when wet. It is important to note that it has been generally recognized that the second strongest natural fiber in the world is cotton and ramie is three times stronger than cotton. Additionally, ramie fiber is approximately 30% stronger when wet, does not stretch and is not affected by ultraviolet rays as for example, nylon would be, which is why ramie is the leading quality rope for marine usage.

Additional testing was performed on ramie fiber using an Uster High Volume Instrumentation Line, wherein the ramie fiber was initially run through a Garnett machine, designed to convert rags back into fiber. The ramie fiber strength averaged 35.4 g/tex or 3.93 g per dernier, a number which is approximately the same as regular tenacity polyester. Fiber fineness for the ramie sample averaged 7.7 micrograms per inch, which is equivalent to a 2.73 denier. Reflectance, as measured as RD part of color was determined to be 71.3 with a B value of 9.7, this value being indicative of the yellow/blue parameters of the material being tested. The higher the number, the more yellow the sample.

Further evidence of the superior characteristics of P.I. 87521-X relate to the planting of this variety in December 1995 in LaBelle, Fla. In February 1996, this area experienced a cold spell, which had not been documented as severe for the past 100 years, the freeze destroying citrus and sugar cane crops, and wherein the temperature never exceeded 40° F. until April 1996 and dropped as low as 22° F. The entire ramie crop survived this disastrous freeze, thereby lending further support for the uniqueness of this plant, which has been transformed from a tropical or subtropical plant into a more cold weather resistant variety.

This invention has been described in detail with reference to specific embodiments thereof, including the respective best modes for carrying out each embodiment. It shall be understood that these illustrations are by way of example and not by way of limitation.

What is claimed is:

1. Seed of a Boehmeria nivea (L) Gaud line designated P.I. 87521-X and having Accession Number (to be assigned).

2. A Boehmeria nivea (L) Gaud plant, or its parts, produced by the seed of claim 1.

3. Pollen of the plant of claim 2.

4. An ovule of the plant of claim 2.

5. A Boehmeria nivea (L) Gaud plant having all the physiological and morphological characteristics of the plant of claim 2.
